(19) United States
(12) Patent Application Publication
(10) Pub. No.: US 2002/0081697 A1
(43) Pub. Date: Jun. 27, 2002

(54) Thermostable enzymes having aminotransferase activity, nucleic acids encoding them and methods of making and using them

(76) Inventors: Ikuo Matsui, Ibaraki (JP); Kazuhiko Ishikawa, Osaka (JP); Hiroyasu Ishida, Ibaraki (JP); Yoshitsugu Kosugi, Ibaraki (JP); Eriko Matsui, Ibaraki (JP)

Correspondence Address: GREGORY P. EINHORN, Fish & Richardson P.C., Suite 500, 4350 La Jolla Village Drive, San Diego, CA, 92122, US

(21) Appl. No.: 09/967,645

(22) Filed: Sep. 28, 2001

Related U.S. Application Data
(63) Continuation-in-part of application No. PCT/JP99/01696, filed on Mar. 31, 1999.

(51) Int. Cl. C12N 9/10; C07H 21/04; C12N 5/06; C12P 21/02

(52) U.S. Cl. 435/193; 435/320.1; 435/325; 435/69.1; 536/23.2

(57) ABSTRACT

The invention is directed to novel thermostable aminotransferases useful in synthesizing an amino acid derivative with high optical purity, and nucleic acids encoding the enzyme. The invention also includes antibodies that specifically bind to the aminotransferases of the invention.

### RELATED APPLICATIONS

This application is a continuation in part (CIP) and claims the benefit of priority under 35 U.S.C. §120 to Patent Convention Treaty (PCT) International Application Serial No: PCT/JP99/01696, filed Mar. 31, 1999. The aforementioned application is explicitly incorporated herein by reference in its entirety and for all purposes.

### TECHNICAL FIELD

The present invention generally relates to the fields of biochemistry and protein synthesis. In particular, the invention is directed to novel thermostable aminotransferases useful in synthesizing an amino acid derivative with high optical purity, and nucleic acids encoding the enzyme.

### BACKGROUND

Aminotransferases are enzymes useful in synthesizing amino acids, amines, and prochiral ketones with high optical purity. Aminotransferases can catalyze a reaction to produce other oxo acids and amino acids by transferring amino groups of amino acids to alpha-keto acids (see FIG. 1). This reaction synthesizes amino acid derivatives retaining stereoisomerism of amino group donors (FIG. 2).

A variety of aminotransferases with different substrate specificities have been isolated from mammalian cells and yeast cells. However, these transferases have poor heat resistance, acid-resistance and alkali-resistance since most of them are derived from mesophilic organisms. Because of such poor resistance, these aminotransferases were not able to be used for chemical synthesis (e.g. amino acid derivative synthesis) under severe conditions in which organic solvents and the like are used.

Therefore, isolation of aminotransferase which remains stable at high temperature and over a wide pH range can provide very useful, novel catalyst in chemical synthesis (e.g. amino acid derivative synthesis) under severe conditions. Consequently, development of aminotransferase which remains stable under severe conditions has been desired.

### SUMMARY OF THE INVENTION

The invention provides an isolated enzyme comprising aminotransferase activity comprising the following properties: (a) the enzyme has molecular weight of between about 43,000 Da (daltons) and about 45,000 Da, or, has an isoelectric point of between about 5.0 and 5.4; and, (b) the enzyme comprises an aminotransferase activity and exhibits higher aminotransferase activity when an aromatic amino acid is used as an amino group donor rather than when a non-aromatic amino acid is used as an amino group donor. In one aspect, the enzyme retains its aminotransferase activity at temperatures over about 90° C. The optimum aminotransferase activity of the enzyme can be at a temperature of about 90° C. The enzyme can have aminotransferase activity in conditions comprising a pH of between about pH 4 to about pH 11. The optimum aminotransferase activity of the enzyme can be at a pH of about pH 6. The enzyme can maintain its activity after exposure to treatment at about pH 6.5 and 95° C. for about 6 hours. The enzyme can remain stable at about pH 4 to about pH 11 and about 25° C. for 24 hours or more. The enzyme can have a melting temperature at about pH 6.5 at about 120.1° C. where molar enthalpy change is about 2.4×103 KJ/mole. The enzyme can have an a-helix content of about 40% at about pH 6.5 and about 25° C. The enzyme can have a molecular weight of about 44,000 Da. The enzyme can have a homodimeric subunit structure. The enzyme can have an isoelectric point of about 5.2. In one aspect, when the enzyme is denatured, the denaturation is an irreversible process. The enzyme can comprise a sequence as set forth in SEQ ID NO:1.

The invention provides an isolated enzyme comprising aminotransferase activity comprising the following properties: (a) the enzyme has molecular weight of about 44,000 Da and an isoelectric point of about 5.2; (b) the enzyme exhibits higher aminotransferase activity when an aromatic amino acid is used as an amino group donor rather than when a non-aromatic amino acid is used as an amino group donor, and, (c) the enzyme has an aminotransferase activity and retains its aminotransferase activity at temperatures over about 90° C.

The invention provides an isolated polypeptide comprising an amino acid sequence as set forth in SEQ ID NO:1.

The invention provides an isolated polypeptide comprising an amino acid sequence derived from the amino acid sequence of SEQ ID NO: 1 further comprising a deletion, a substitution or an addition of one or more amino acid residues of SEQ ID NO: 1 and having an aminotransferase activity. The substitutions can be conservative substitutions, for example, a hydrophobic residue or a hydrophobic residue, a charged residue for a similarly charged residue, and the like.

The invention provides an isolated polypeptide comprising an amino acid sequence having at least 85% sequence identity to SEQ ID NO:1, and, the polypeptide has an aminotransferase activity. In alternative aspects, the sequence identity to SEQ ID NO:1 is at least 80%, at least 85%, at least 90%, at least 95%, at least 98%, at least 99%. The polypeptide can have a sequence as set forth in SEQ ID NO:1.

The invention provides an isolated nucleic acid, wherein the nucleic acid encodes a polypeptide of the invention. The invention provides an isolated nucleic acid, wherein the nucleic acid hybridizes under stringent hybridization conditions to an aminotransferase-encoding nucleic acid of the invention, e.g., the exemplary nucleic acid of the invention, as set forth in SEQ ID NO:2. The invention provides an isolated nucleic acid comprising a sequence having at least 85% sequence identity to SEQ ID NO:2, and, the polypeptide encoded by this nucleic acid has an aminotransferase activity. In alternative aspects, the sequence identity to SEQ ID NO:2 is at least 80%, at least 85%, at least 90%, at least 95%, at least 98%, at least 99%. The invention provides an isolated nucleic acid, wherein the nucleic acid encodes a polypeptide as set forth in SEQ ID NO:1.

The invention provides an expression cassette comprising a nucleic acid of the invention. The expression cassette can be, e.g., a plasmid, a recombinant virus, a naked DNA operatively linked to a promoter, and the like. The invention provides a transformed cell comprising a heterologous nucleic acid, wherein the heterologous nucleic acid comprises a sequence of the invention. The invention provides an array comprising oligonucleotide probes immobilized on a solid support comprising a nucleic acid of the invention. The invention provides an array comprising polypeptides immobilized on a solid support comprising a polypeptide of the invention.

The invention provides an isolated antibody that selectively binds to a polypeptide of the invention, or a polypeptide encoded by a nucleic acid of the invention. The antibody can be a polyclonal or a monoclonal antibody. The invention provides a hybridoma cell line comprising an antibody of the invention.

The invention provides a method of making a transformed cell comprising a heterologous aminotransferase nucleic acid or polypeptide comprising introducing a nucleic acid of the invention into a cell, thereby producing a transformed cell.

The invention provides a method of expressing a heterologous nucleic acid sequence in a cell comprising: (a) transforming the cell with a heterologous nucleic acid sequence comprising a nucleic acid of the invention, wherein heterologous nucleic acid sequence comprises a promoter operably linked to the nucleic acid sequence; and, (b) growing the cell under conditions where the heterologous nucleic acid sequence is expressed in the cell.

The invention provides a method of determining whether a test compound specifically binds to an aminotransferase enzyme comprising: (a) expressing a nucleic acid of the invention under conditions permissive for translation of the nucleic acid to a polypeptide, or, providing a polypeptide of the invention; (ii) contacting the polypeptide with the test compound; and, (iii) determining whether the test compound specifically binds to the polypeptide.

The details of one or more embodiments of the invention are set forth in the accompanying drawings and the description below. Other features, objects, and advantages of the invention will be apparent from the description and drawings, and from the claims.

All publications, patents, patent applications, GenBank sequences and ATCC deposits, cited herein are hereby expressly incorporated by reference for all purposes.

### BRIEF DESCRIPTION OF THE DRAWINGS

FIG. 1 is a schematic diagram showing how aminotransferases can catalyze a reaction to produce other oxo acids and amino acids by transferring amino groups of amino acids to alpha-keto acids.

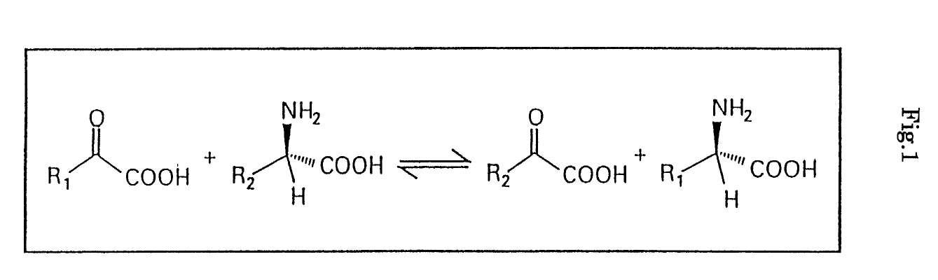

FIG. 2 is a schematic diagram showing how an aminotransferase reaction synthesizes amino acid derivatives retaining stereoisomerism of amino group donors.

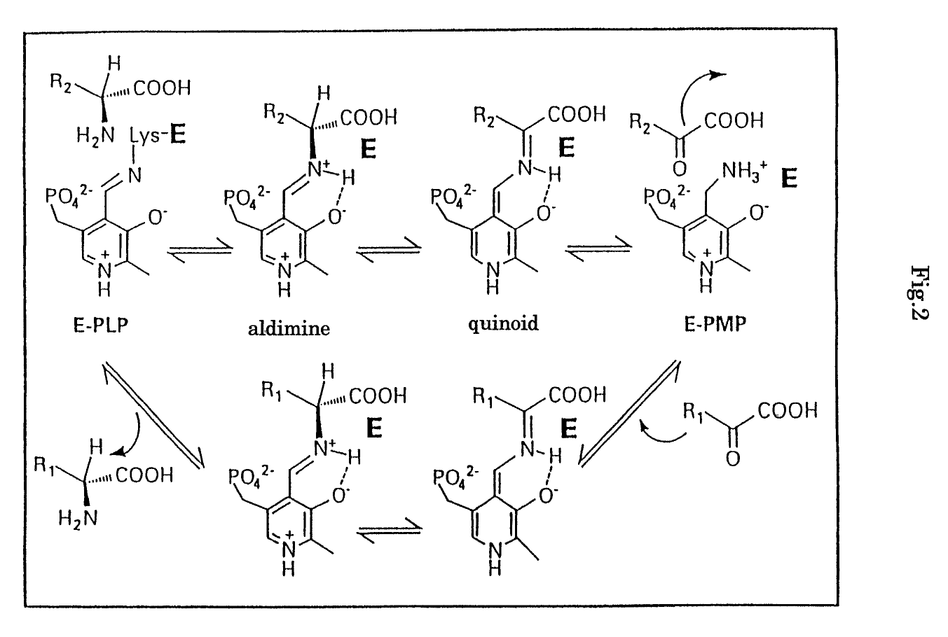

FIG. 3 is a graphic summary of the pH dependence of the Kapp value of an enzyme of the invention, as described in detail in Example 9, below.

### DETAILED DESCRIPTION

The present invention provides a novel aminotransferase that remains stable at high temperature and over a wide pH range. Also provided are nucleic acids, e.g., genes, encoding the same, expression cassettes and transformed cells comprising the nucleic acids of the invention, and antibodies the specifically bind to the enzymes of the invention.

As a result of thorough studies to address the above problems, the present inventors have determined a nucleotide sequence of a chromosomal DNA of an extreme thermophilic bacterium capable of growing at 90° C. to 100° C. Based on that nucleotide sequence, the present inventors have isolated a gene that encodes a protein having aminotransferase activity. The present inventors have also integrating the gene into a bacterium, e.g., E. coli, for expression and to confirm that the protein encoded by the gene has aminotransferase activity, and remains stable and has aminotransferase activity at high temperatures of about 90° C. or more, and has aminotransferase activity over a wide pH range, from about pH 4 to pH 11.

In one aspect, the present invention is an enzyme which: has aminotransferase activity, exhibits higher aminotransferase activity when an aromatic amino acid is used as an amino group donor rather than when a non-aromatic amino acid is used as an amino group donor, and, has an optimum temperature of about 90° C.

In one aspect, the present invention is an enzyme which has aminotransferase activity, exhibits higher aminotransferase activity when an aromatic amino acid is used as an amino group donor than when a non-aromatic amino acid is used as an amino group donor, has an optimum temperature of about 90° C., has an optimum about pH of about 6.0, maintains its activity even when subjected to treatment at pH 6.5 and about 95° C. for 6 hours, has a half-life at about pH 6.5 and about 110° C. of about 30 minutes, remains stable at about pH 4 to about pH 11 and about 25° C. for about 24 hours or more, has a melting temperature at about pH 6.5 of about 120.1° C. where molar enthalpy change is about 2.4×103 KJ/mole, has an a-helix content of about 40% at about pH 6.5 and about 25° C., has molecular weight of about 44,000 Da, has a homodimeric subunit structure, has an isoelectric point of about 5.2, and for which denaturation is irreversible.

In one aspect, the present invention is a protein which is the following protein (a) or (b): (a) a protein which comprises an amino acid sequence of SEQ ID NO: 1, (b) a protein which comprises an amino acid sequence derived from the amino acid sequence of SEQ ID NO: 1 by deletion, replacement or addition of one or more amino acids and has aminotransferase activity.

In another aspect, the present invention provides a nucleic acid, e.g., a gene, which encodes the protein as set forth above.

The enzyme of this invention can be obtained, for example, by the following exemplary methods. The cells of a microorganism capable of producing the enzyme of this invention are disrupted, suspended in a buffer, and then centrifuged. The supernatant obtained by the centrifugation is purified by a variety of chromatography based on the presence of aminotransferase activity as an index. Thus, the enzyme of this invention can be obtained.

A buffer and conditions for centrifugation and chromatography employed in the above methods may be appropriately selected from a normal range employed upon purification of enzymes from microbial cells.

The presence of aminotransferase activity can be determined by any means. One example, aminotransferase activity is determined by tracing an increase in absorbance at 412 nm resulting from reduction of 5,5′-Dithiobis (2-nitrobenzoic acid)(DTNB) with L-cysteic acid and 2-ketoglutaric acid as substrates.

Any microorganism or expression system (including yeast, plant, insect or mammalian) can be employed in the above methods. That is, all microorganisms, yeast, plant, insect or mammalian cells are employed in practicing the methods and making the compositions of the invention as long as they can produce the enzyme of this invention (e.g., by recombinant methods).

For example, an extreme thermophilic bacterium can be used. Exemplary thermophilic bacterium include the sulfur-metabolizing thermophilic archaebacterium, Pyrococcus horikoshi (deposited at JAPAN Collection of Microorganism, RIKEN, Accession No.: JCM9974) can be used. In addition, a microorganism (e.g. E. coli) into which the gene of this invention has been transferred as described below can be used.

The enzyme of this invention has aminotransferase activity, and remains stable at high temperature and over a wide pH range so that it can be used as a catalyst for aminotransferase reaction under severe conditions. With very high amino transferase activity for an aromatic amino acid, the enzyme of this invention is particularly useful as a catalyst for aminotransferase reaction using an aromatic amino acid as a substrate. Aminotransferase reactions using the enzyme of this invention can provide an amino acid derivative with high optical purity.

In one aspect, the invention provides the following protein (a) or (b): (a) a protein which comprises an amino acid sequence of SEQ ID NO: 1, (b) a protein which comprises an amino acid sequence derived from the amino acid sequence of SEQ ID NO: 1 by deletion, replacement or addition of one or more amino acids and has aminotransferase activity. Here, the number of amino acids represented by “one or more amino acids” is not specifically limited as long as they are deleted, replaced or added by techniques standard at the time when the present application is filed and do not lose aminotransferase activity. Further, a protein in which one or more amino acids are deleted, replaced or added can be produced by techniques standard at the time when the present application was filed, e.g. site-directed mutagenesis (see, e.g., Zoller et al., Nucleic acids Res. 10, 6487-6500, 1982).

A protein of this invention can be obtained by the same steps as employed for the enzyme of this invention. As with the enzyme of this invention, the protein of this invention has aminotransferase activity in addition to stability at high temperature and over a wide pH range. Hence, the protein of this invention can be used as a catalyst for aminotransferase reaction under severe conditions. Further, like the enzyme of the present invention, the protein of the present invention has very high aminotransferase activity for an aromatic amino acid, and is particularly useful as a catalyst for aminotransferase reaction using an aromatic amino acid as a substrate. Like the enzyme of this invention, aminotransferase reaction using the protein of this invention can provide an amino acid derivative with high optical purity.

In another aspect, the invention provides nucleic acids, e.g., isolated or cloned nucleic acids, isolated or cloned genes, transcripts, cDNAs, recombinantly produced nucleic acids, encoding the protein of the invention. For example, the nucleic acids of this invention can be obtained as described below.

In one exemplary protocol, chromosomal DNA can be extracted from microorganisms having the gene of this invention. Microorganisms used herein are not specifically limited as long as they have the gene of this invention. Examples of such a microorganism include extreme thermophilic bacteria. More specifically, a sulfur-metabolizing thermophilic archaebacterium, Pyrococcus horikoshi (deposited at JAPAN Collection of Microorganism, RIKEN, Accession No: JCM9974) can be used. In addition, chromosomal DNA can be extracted from microorganisms by standard techniques.

Next, the extracted chromosomal DNA is partially digested with restriction enzymes and then inserted into a vector. Restriction enzymes used herein are not specifically limited as long as they can cleave chromosomal DNA to appropriate lengths, such as a length of approximately 40 kb. Examples of such restriction enzymes include, but are not limited to, HindIII, EcoRI, SalI, and KpnI. A preferable restriction enzyme is HindIII. A vector used herein is not specifically limited as long as it can function as a cloning vector. Examples of such vectors include pBAC108L and pFOS1.

Subsequently, the above recombinant vector is introduced into an appropriate host cell to construct a genome DNA library, followed by determination of the nucleotide sequence of chromosomal DNA. Examples of the host cell which can be used herein include, but are not limited to, Escherichia coli and yeast cells. A method for introducing a recombinant vector into a host cell may be appropriately selected depending on the vector to be used. For example, electroporation is preferred when pBAC108L is used as a vector; 1 phage or the like is preferred when pFOS 1 is used as a vector. Further, the nucleotide sequence of chromosomal DNA can be determined by for example, Maxim-Gilbert chemical modification method, dideoxynucleotide chain termination, or modified methods therefrom which are automated. Then, homologous regions of the protein of this invention are found from the obtained sequence data, and a structural gene encoding the protein of this invention is identified. Next, primers complementary to both ends of the structural gene above are synthesized and used for PCR to amplify the structural gene so that the gene of this invention can be obtained.

In another aspect, the nucleic acids of this invention can be chemically synthesized by a known method such as the phosphite triester method.

Escherichia coli BL21 PET11a/ArATph into which the gene of this invention is transferred was internationally deposited as FERM BP-6685 at the National Institute of Advanced Industrial Science and Technology (AIST) (1-1-3, Higashi, Tsukuba, Ibaraki, JAPAN) under the Budapest Treaty (deposition date: Jan. 26, 1998).

The nucleic acids of this invention encode the protein of this invention. That is, the nucleic acids of this invention can be integrated into an expression vector, and the vector is introduced into and expressed in a host cell derived from a prokaryotic or eukaryotic organism, thereby producing the protein of this invention in large quantity. Examples of the expression vectors which can be used herein include pET11a and pET15b. Examples of a host cell derived from prokaryotic organism include E. coli (e.g. E. coli BL21 (DE3), E. coli XL1-BlueMRF′ and the like) and Bacillus subtilis. Examples of a host cell derived from a eukaryotic organism that can be used herein include a vertebrate cell and a yeast cell.

Definitions

Unless defined otherwise, all technical and scientific terms used herein have the meaning commonly understood by a person skilled in the art to which this invention belongs. As used herein, the following terms have the meanings ascribed to them unless specified otherwise.

The term “antibody” or “Ab” includes both intact antibodies having at least two heavy (H) chains and two light (L) chains inter-connected by disulfide bonds and antigen binding fragments thereof, or equivalents thereof, either isolated from natural sources, recombinantly generated or partially or entirely synthetic. Examples of antigen binding fragments include, e.g., Fab fragments, F(ab′)2 fragments, Fd fragments, dAb fragments, isolated complementarity determining regions (CDR), single chain antibodies, chimeric antibodies, humanized antibodies, human antibodies made in non-human animals (e.g., transgenic mice) or any form of antigen binding fragment.

The terms “array” or “microarray” or “DNA array” or “nucleic acid array” or “biochip” as used herein is a plurality of target elements, each target element comprising a defined amount of one or more nucleic acid and/or polypeptide molecules, including the nucleic acids and polypeptides of the invention, immobilized a solid surface for hybridization to sample nucleic acids, as described in detail, below. The nucleic acids of the invention can be incorporated into any form of microarray, as described, e.g., in U.S. Pat. Nos. 6,045,996; 6,022,963; 6,013,440; 5,959,098; 5,856,174; 5,770,456; 5,556,752; 5,143,854.

The term “expression cassette” as used herein refers to a nucleotide sequence which is capable of affecting expression of a structural gene (i.e., a protein coding sequence) in a host compatible with such sequences. Expression cassettes include at least a promoter operably linked with the polypeptide coding sequence; and, optionally, with other sequences, e.g., transcription termination signals. Additional factors necessary or helpful in effecting expression may also be used, e.g., enhancers. “Operably linked” as used herein refers to linkage of a promoter upstream from a DNA sequence such that the promoter mediates transcription of the DNA sequence. Thus, expression cassettes also include plasmids, expression vectors, recombinant viruses, any form of recombinant “naked DNA” vector, and the like. A “vector” comprises a nucleic acid that can infect, transfect, transiently or permanently transduce a cell. It will be recognized that a vector can be a naked nucleic acid, or a nucleic acid complexed with protein or lipid. The vector optionally comprises viral or bacterial nucleic acids and/or proteins, and/or membranes (e.g., a cell membrane, a viral lipid envelope, etc.). Vectors include, but are not limited to replicons (e.g., RNA replicons, bacteriophages) to which fragments of DNA may be attached and become replicated. Vectors thus include, but are not limited to RNA, autonomous self-replicating circular or linear DNA or RNA (e.g., plasmids, viruses, and the like, see, e.g., U.S. Pat. No. 5,217,879), and includes both the expression and nonexpression plasmids. Where a recombinant microorganism or cell culture is described as hosting an “expression vector” this includes both extrachromosomal circular and linear DNA and DNA that has been incorporated into the host chromosome(s). Where a vector is being maintained by a host cell, the vector may either be stably replicated by the cells during mitosis as an autonomous structure, or is incorporated within the host's genome.

The term “isolated” as used herein, when referring to a molecule or composition, such as, e.g., a nucleic acid or polypeptide of the invention, means that the molecule or composition is separated from at least one other compound, such as a protein, other nucleic acids (e.g., RNAs), or other contaminants with which it is associated in vivo or in its naturally occurring state. Thus, a nucleic acid or polypeptide is considered isolated when it has been isolated from any other component with which it is naturally associated, e.g., cell membrane, as in a cell extract. An isolated composition can, however, also be substantially pure. An isolated composition can be in a homogeneous state and can be in a dry or an aqueous solution. Purity and homogeneity can be determined, for example, using analytical chemistry techniques such as polyacrylamide gel electrophoresis (SDS-PAGE) or high performance liquid chromatography (HPLC). Thus, the isolated compositions of this invention do not contain materials normally associated with their in situ environment. Even where a protein has been isolated to a homogenous or dominant band, there can be trace contaminants which co-purify with the desired protein.

The term “nucleic acid” or “nucleic acid sequence” refers to a deoxy-ribonucleotide or ribonucleotide oligonucleotide, including single- or double-stranded forms, and coding or non-coding (e.g., “antisense”) forms. The term encompasses nucleic acids containing known analogues of natural nucleotides. The term also encompasses nucleic-acid-like structures with synthetic backbones. DNA backbone analogues provided by the invention include phosphodiester, phosphorothioate, phosphorodithioate, methylphosphonate, phosphoramidate, alkyl phosphotriester, sulfamate, 3′-thioacetal, methylene(methylimino), 3′-N-carbamate, morpholino carbamate, and peptide nucleic acids (PNAs); see Oligonucleotides and Analogues, a Practical Approach, edited by F. Eckstein, IRL Press at Oxford University Press (1991); Antisense Strategies, Annals of the New York Academy of Sciences, Volume 600, Eds. Baserga and Denhardt (NYAS 1992); Milligan (1993) J. Med. Chem. 36:1923-1937; Antisense Research and Applications (1993, CRC Press). PNAs contain non-ionic backbones, such as N-(2-aminoethyl) glycine units. Phosphorothioate linkages are described, e.g., by U.S. Pat. Nos. 6,031,092; 6,001,982; 5,684,148; see also, WO 97/03211; WO 96/39154; Mata (1997) Toxicol. Appl. Pharmacol. 144:189-197. Other synthetic backbones encompassed by the term include methyl-phosphonate linkages or alternating methylphosphonate and phosphodiester linkages (see, e.g., U.S. Pat. No. 5,962,674; Strauss-Soukup (1997) Biochemistry 36:8692-8698), and benzylphosphonate linkages (see, e.g., U.S. Pat. No. 5,532,226; Samstag (1996) Antisense Nucleic Acid Drug Dev 6:153-156). The term nucleic acid is used interchangeably with gene, DNA, RNA, cDNA, mRNA, oligonucleotide primer, probe and amplification product.

As used herein the terms “polypeptide,” “protein,” and “peptide” are used interchangeably and include compositions of the invention that also include “analogs,” or “conservative variants” and “mimetics” (e.g., “peptidomimetics”) with structures and activity that substantially correspond to the polypeptides of the invention, including the exemplary sequence as set forth herein. Thus, the terms “conservative variant” or “analog” or “mimetic” also refer to a polypeptide or peptide which has a modified amino acid sequence, such that the change(s) do not substantially alter the polypeptide's (the conservative variant's) structure and/or activity (e.g., aminotransferase activity), as defined herein. These include conservatively modified variations of an amino acid sequence, i.e., amino acid substitutions, additions or deletions of those residues that are not critical for protein activity, or substitution of amino acids with residues having similar properties (e.g., acidic, basic, positively or negatively charged, polar or non-polar, etc.) such that the substitutions of even critical amino acids does not substantially alter structure and/or activity. Conservative substitution tables providing functionally similar amino acids are well known in the art. For example, one exemplary guideline to select conservative substitutions includes (original residue followed by exemplary substitution): ala/gly or ser; arg/lys; asn/gln or his; asp/glu; cys/ser; gln/asn; gly/asp; gly/ala or pro; his/asn or gln; ile/leu or val; leu/ile or val; lys/arg or gln or glu; met/leu or tyr or ile; phe/met or leu or tyr; ser/thr; thr/ser; trp/tyr; tyr/trp or phe; val/ile or leu. An alternative exemplary guideline uses the following six groups, each containing amino acids that are conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W); (see also, e.g., Creighton (1984) Proteins, W. H. Freeman and Company; Schulz and Schimer (1979) Principles of Protein Structure, Springer-Verlag). One of skill in the art will appreciate that the above-identified substitutions are not the only possible conservative substitutions. For example, for some purposes, one may regard all charged amino acids as conservative substitutions for each other whether they are positive or negative. In addition, individual substitutions, deletions or additions that alter, add or delete a single amino acid or a small percentage of amino acids in an encoded sequence can also be considered “conservatively modified variations.”

The terms “mimetic” and “peptidomimetic” refer to a synthetic chemical compound that has substantially the same structural and/or functional characteristics of the polypeptides of the invention (e.g., aminotransferase activity). The mimetic can be either entirely composed of synthetic, non-natural analogues of amino acids, or, is a chimeric molecule of partly natural peptide amino acids and partly non-natural analogs of amino acids. The mimetic can also incorporate any amount of natural amino acid conservative substitutions as long as such substitutions also do not substantially alter the mimetics' structure and/or activity. As with polypeptides of the invention which are conservative variants, routine experimentation will determine whether a mimetic is within the scope of the invention, i.e., that its structure and/or function is not substantially altered. Polypeptide mimetic compositions can contain any combination of non-natural structural components, which are typically from three structural groups: a) residue linkage groups other than the natural amide bond (“peptide bond”) linkages; b) non-natural residues in place of naturally occurring amino acid residues; or c) residues which induce secondary structural mimicry, i.e., to induce or stabilize a secondary structure, e.g., a beta turn, gamma turn, beta sheet, alpha helix conformation, and the like. A polypeptide can be characterized as a mimetic when all or some of its residues are joined by chemical means other than natural peptide bonds. Individual peptidomimetic residues can be joined by peptide bonds, other chemical bonds or coupling means, such as, e.g., glutaraldehyde, N-hydroxysuccinimide esters, bifunctional maleimides, N,N′-dicyclohexylcarbodiimide (DCC) or N,N′-diisopropylcarbodiimide (DIC). Linking groups that can be an alternative to the traditional amide bond (“peptide bond”) linkages include, e.g., ketomethylene (e.g., —C(═O)—CH2— for —C(═O)—NH—), aminomethylene (CH2—NH), ethylene, olefin (CH═CH), ether (CH2—O), thioether (CH2—S), tetrazole (CN4—), thiazole, retroamide, thioamide, or ester (see, e.g., Spatola (1983) in Chemistry and Biochemistry of Amino Acids, Peptides and Proteins, Vol. 7, pp 267-357, “Peptide Backbone Modifications,” Marcell Dekker, NY). A polypeptide can also be characterized as a mimetic by containing all or some non-natural residues in place of naturally occurring amino acid residues; non-natural residues are well described in the scientific and patent literature.

The term percent “sequence identity,” in the context of two or more nucleic acids or polypeptide sequences refers to two or more sequences or subsequences that are the same or have a specified percentage of nucleotides (or amino acid residues) that are the same, when compared and aligned for maximum correspondence over a comparison window, as measured using one of the following sequence comparison algorithms or by manual alignment and visual inspection. This definition also refers to the complement (antisense strand) of a sequence. For example, in alternative embodiments, nucleic acids within the scope of the invention include those with a nucleotide sequence identity that is at least about 95%, at least about 96%, at least about 97%, at least about 98%, at least about 99% of the exemplary sequence set forth in SEQ ID NO:2. In alternative embodiments, polypeptides within the scope of the invention include those with an amino acid sequence identity that is least about 80%, least about 85%, least about 90%, at least about 95%, at least about 96%, at least about 97%, at least about 98%, at least about 99% of the exemplary sequences set forth in SEQ ID NO:1. Two sequences with these levels of identity are “substantially identical” and within the scope of the invention. Thus, if a nucleic acid sequence has the requisite sequence identity to SEQ ID NO:2, or a subsequence thereof, it also is a polynucleotide sequence within the scope of the invention. If a polynucleotide sequence has the requisite sequence identity to SEQ ID NO:2, or a subsequence thereof, it also is a polypeptide within the scope of the invention. In one aspect, the percent identity exists over a region of the sequence that is at least about 25 nucleotides or amino acid residues in length, or, over a region that is at least about 50 to 100 nucleotides or amino acids in length. Parameters (including, e.g., window sizes, gap penalties and the like) to be used in calculating “percent sequence identities” between two nucleic acids or polypeptides to identify and determine whether one is within the scope of the invention are described in detail, below.

The phrase “selectively (or specifically) hybridizes to” refers to the binding, duplexing, or hybridizing of a molecule to a particular nucleotide sequence under stringent hybridization conditions when that sequence is present in a complex mixture (e.g., total cellular or library DNA or RNA), wherein the particular nucleotide sequence is detected at least at about 10 times background. In one embodiment, a nucleic acid can be determined to be within the scope of the invention (e.g., is substantially identical to SEQ ID NO:2) by its ability to hybridize under stringent conditions to a nucleic acid otherwise determined to be within the scope of the invention (such as the exemplary sequences described herein).

The phrase “stringent hybridization conditions” refers to conditions under which a probe will primarily hybridize to its target subsequence, typically in a complex mixture of nucleic acid, but to no other sequences in significant amounts, is described in detail below. A positive signal (e.g., identification of a nucleic acid of the invention) is about 10 times background hybridization.

“Stringent” hybridization conditions that are used to identify substantially identical nucleic acids within the scope of the invention include hybridization in a buffer comprising 50% formamide, 5× SSC, and 1% SDS at 42° C., or hybridization in a buffer comprising 5× SSC and 1% SDS at 65° C., both with a wash of 0.2× SSC and 0.1% SDS at 65° C. Exemplary “moderately stringent hybridization conditions” include a hybridization in a buffer of 40% formamide, 1 M NaCl, and 1% SDS at 37° C., and a wash in 1× SSC at 45° C. Those of ordinary skill will readily recognize that alternative but comparable hybridization and wash conditions can be utilized to provide conditions of similar stringency. Nucleic acids which do not hybridize to each other under moderately stringent or stringent hybridization conditions are still substantially identical if the polypeptides which they encode are substantially identical. This may occur, e.g., when a copy of a nucleic acid is created using the maximum codon degeneracy permitted by the genetic code, as discussed herein (see discussion on “conservative substitutions”). However, the selection of a hybridization format is not critical—it is the stringency of the wash conditions that set forth the conditions which determine whether a nucleic acid is within the scope of the invention. Wash conditions used to identify nucleic acids within the scope of the invention include, e.g.: a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2× SSC at a temperature of at least about 50° C. or about 55° C. to about 60° C. for about 15 to about 20 minutes; or, the hybridization complex is washed twice with a solution with a salt concentration of about 2× SSC containing 0.1% SDS at room temperature for 15 minutes and then washed twice by 0.1× SSC containing 0.1% SDS at 68° C. for 15 minutes; or, equivalent conditions. See Sambrook, Tijssen and Ausubel for a description of SSC buffer and equivalent conditions.

Polypeptides and Peptides

The invention provides an isolated or recombinant polypeptide comprising a sequence having various sequence identities to SEQ ID NO:1, as set forth above. One exemplary polypeptide comprises the sequence as set forth in SEQ ID NO:1, and fragments (e.g., antigenic fragments) thereof (as noted above, the term polypeptide includes peptides and peptidomimetics, etc.). Polypeptides and peptides of the invention can be isolated from natural sources, be synthetic, or be recombinantly generated polypeptides. Peptides and proteins can be recombinantly expressed in vitro or in vivo. The peptides and polypeptides of the invention can be made and isolated using any method known in the art.

Polypeptide and peptides of the invention can also be synthesized, whole or in part, using chemical methods well known in the art. See e.g., Caruthers (1980) Nucleic Acids Res. Symp. Ser. 215-223; Horn (1980) Nucleic Acids Res. Symp. Ser. 225-232; Banga, A. K., Therapeutic Peptides and Proteins, Formulation, Processing and Delivery Systems (1995) Technomic Publishing Co., Lancaster, Pa. For example, peptide synthesis can be performed using various solid-phase techniques (see e.g., Roberge (1995) Science 269:202; Merrifield (1997) Methods Enzymol. 289:3-13) and automated synthesis may be achieved, e.g., using the ABI 431A Peptide Synthesizer (Perkin Elmer). The skilled artisan will recognize that individual synthetic residues and polypeptides incorporating mimetics can be synthesized using a variety of procedures and methodologies, which are well described in the scientific and patent literature, e.g., Organic Syntheses Collective Volumes, Gilman, et al. (Eds) John Wiley & Sons, Inc., NY. Polypeptides incorporating mimetics can also be made using solid phase synthetic procedures, as described, e.g., by Di Marchi, et al., U.S. Pat. No. 5,422,426. Peptides and peptide mimetics of the invention can also be synthesized using combinatorial methodologies. Various techniques for generation of peptide and peptidomimetic libraries are well known, and include, e.g., multipin, tea bag, and split-couple-mix techniques; see, e.g., al-Obeidi (1998) Mol. Biotechnol. 9:205-223; Hruby (1997) Curr. Opin. Chem. Biol. 1:114-119; Ostergaard (1997) Mol. Divers. 3:17-27; Ostresh (1996) Methods Enzymol. 267:220-234. Modified peptides of the invention can be further produced by chemical modification methods, see, e.g., Belousov (1997) Nucleic Acids Res. 25:3440-3444; Frenkel (1995) Free Radic. Biol. Med. 19:373-380; Blommers (1994) Biochemistry 33:7886-7896.

The invention provides a fusion protein comprising a polypeptide of the invention, and a second domain. Thus, peptides and polypeptides of the invention are synthesized and expressed as chimeric or “fusion” proteins with one or more additional domains linked thereto for, e.g., to more readily isolate or identify a recombinantly synthesized peptide, and the like. Detection and purification facilitating domains include, e.g., metal chelating peptides such as polyhistidine tracts and histidine-tryptophan modules that allow purification on immobilized metals, protein A domains that allow purification on immobilized immunoglobulin, and the domain utilized in the FLAGS extension/affinity purification system (Immunex Corp, Seattle Wash.). The inclusion of a cleavable linker sequences such as Factor Xa or enterokinase (Invitrogen, San Diego Calif.) between the purification domain and GCA-associated peptide or polypeptide can be useful to facilitate purification. For example, an expression vector can include an epitope-encoding nucleic acid sequence linked to six histidine residues followed by a thioredoxin and an enterokinase cleavage site (see, e.g., Williams (1995) Biochemistry 34:1787-1797; Dobeli (1998) Protein Expr. Purif. 12:404-14). The histidine residues facilitate detection and purification while the enterokinase cleavage site provides a means for purifying the epitope from the remainder of the fusion protein.

Nucleic Acids, Expression Vectors and Transformed Cells

The invention provides an isolated or recombinant nucleic acid comprising a nucleic acid sequence having at least 95% sequence identity to SEQ ID NO:2, and expression cassettes (e.g., vectors), cells and transgenic animals comprising the nucleic acids of the invention. As the genes and vectors of the invention can be made and expressed in vitro or in vivo, the invention provides for a variety of means of making and expressing these genes and vectors. One of skill will recognize that desired phenotypes associated with altered gene activity can be obtained by modulating the expression or activity of the genes and nucleic acids (e.g., promoters) within the expression cassettes (e.g., vectors) of the invention. Any of the known methods described for increasing or decreasing expression or activity can be used for this invention. The invention can be practiced in conjunction with any method or protocol known in the art, which are well described in the scientific and patent literature.

The nucleic acid sequences of the invention and other nucleic acids used to practice this invention, whether RNA, cDNA, genomic DNA, vectors, viruses or hybrids thereof, may be isolated from a variety of sources, genetically engineered, amplified, and/or expressed recombinantly. Any recombinant expression system can be used, including, in addition to insect and bacterial cells, e.g., mammalian, yeast or plant cell expression systems.

Alternatively, these nucleic acids can be synthesized in vitro by well-known chemical synthesis techniques, as described in, e.g., Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066.

Techniques for the manipulation of nucleic acids, such as, e.g., generating mutations in sequences, subcloning, labeling probes, sequencing, hybridization and the like are well described in the scientific and patent literature, see, e.g., Sambrook, ed., MOLECULAR CLONING: A LABORATORY MANUAL (2ND ED.), Vols. 1-3, Cold Spring Harbor Laboratory, (1989); CURRENT PROTOCOLS IN MOLECULAR BIOLOGY, Ausubel, ed. John Wiley & Sons, Inc., New York (1997); LABORATORY TECHNIQUES IN BIOCHEMISTRY AND MOLECULAR BIOLOGY: HYBRIDIZATION WITH NUCLEIC ACID PROBES, Part I. Theory and Nucleic Acid Preparation, Tijssen, ed. Elsevier, N.Y. (1993).

The invention provides nucleic acids of the invention “operably linked” to a transcriptional regulatory sequence. “Operably linked” refers to a functional relationship between two or more nucleic acid (e.g., DNA) segments. Typically, it refers to the functional relationship of a transcriptional regulatory sequence to a transcribed sequence. For example, a promoter is operably linked to a coding sequence, such as a nucleic acid of the invention, if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance. For example, in one embodiment, a promoter is operably linked to a nucleic acid sequence of the invention.

The invention further provides cis-acting transcriptional regulatory sequences, which, in vivo, are operably linked to the coding sequence for the exemplary polypeptide of the invention, SEQ ID NO:1, including promoters, comprising the genomic sequences 5′ (upstream) of a transcriptional start site and intronic sequences. The promoters of the invention contain cis-acting transcriptional regulatory elements involved in message expression. These promoter sequences may be readily obtained using routine molecular biological techniques. For example, additional genomic (and promoter) sequences may be obtained by screening Bombyx mori genomic libraries using nucleic acids of the invention. For example, genomic sequence can be readily identified by “chromosome walking” techniques, as described by, e.g., Hauser (1998) Plant J 16:117-125; Min (1998) Biotechniques 24:398-400. Other useful methods for further characterization of promoter sequences include those general methods described by, e.g., Pang (1997) Biotechniques 22:1046-1048; Gobinda (1993) PCR Meth. Applic. 2:318; Triglia (1988) Nucleic Acids Res. 16:8186; Lagerstrom (1991) PCR Methods Applic. 1:111; Parker (1991) Nucleic Acids Res. 19:3055. As is apparent to one of ordinary skill in the art, these techniques can also be applied to identify, characterize and isolate any genomic or cis-acting regulatory sequences corresponding to or associated with the nucleic acid and polypeptide sequences of the invention.

The invention provides oligonucleotide primers that can amplify all or any specific region within a nucleic acid sequence of the invention, particularly, the exemplary SEQ ID NO:2. The nucleic acids of the invention can also be mutated, detected, generated or measured quantitatively using amplification techniques. Using the nucleic acid sequences of the invention (e.g., as in the exemplary SEQ ID NO:2), the skilled artisan can select and design suitable oligonucleotide amplification primers. Amplification methods are also known in the art, and include, e.g., polymerase chain reaction, PCR (see, e.g., PCR PROTOCOLS, A GUIDE TO METHODS AND APPLICATIONS, ed. Innis, Academic Press, N.Y. (1990) and PCR STRATEGIES (1995), ed. Innis, Academic Press, Inc., N.Y.); ligase chain reaction (LCR) (see, e.g., Barringer (1990) Gene 89:117); transcription amplification (see, e.g., Kwoh (1989) Proc. Natl. Acad. Sci. USA, 86:1173); and, self-sustained sequence replication (see, e.g., Guatelli (1990) Proc. Natl. Acad. Sci. USA, 87:1874); Q Beta replicase amplification (see, e.g., Smith (1997) J. Clin. Microbiol. 35:1477-1491; Burg (1996) Mol. Cell. Probes 10:257-271) and other RNA polymerase mediated techniques (e.g., NASBA, Cangene, Mississauga, Ontario).

Expression vectors capable of expressing the nucleic acids and polypeptides of the invention in animal cells, including insect and mammalian cells, are well known in the art. Vectors which may be employed include recombinantly modified enveloped or non-enveloped DNA and RNA viruses, e.g., from baculoviridiae, parvoviridiae, picomoviridiae, herpesveridiae, poxviridae, adenoviridiae, picornnaviridiae or alphaviridae. Insect cell expression systems commonly use recombinant variations of baculoviruses and other nucleopolyhedrovirus, e.g., Bombyx mori nucleopolyhedrovirus vectors (see, e.g., Choi (2000) Arch. Virol. 145:171-177). For example, Lepidopteran and Coleopteran cells are used to replicate baculoviruses to promote expression of foreign genes carried by baculoviruses, e.g., Spodoptera frugiperda cells are infected with recombinant Autographa californica nuclear polyhedrosis viruses (AcNPV) carrying a heterologous, e.g., a human, coding sequence (see, e.g., Lee (2000) J. Virol. 74:11873-11880; Wu (2000) J. Biotechnol. 80:75-83). See, e.g., U.S. Pat. No. 6,143,565, describing use of the polydnavirus of the parasitic wasp Glyptapanteles indiensis to stably integrate nucleic acid into the genome of Lepidopteran and Coleopteran insect cell lines. See also, U.S. Pat. Nos. 6,130,074; 5,858,353; 5,004,687.

Mammalian expression vectors can be derived from adenoviral, adeno-associated viral or retroviral genomes. Retroviral vectors can include those based upon murine leukemia virus (see, e.g., U.S. Pat. No. 6,132,731), gibbon ape leukemia virus (see, e.g., U.S. Pat. No. 6,033,905), simian immuno-deficiency virus, human immuno-deficiency virus (see, e.g., U.S. Pat. No. 5,985,641), and combinations thereof. Describing adenovirus vectors, see, e.g., U.S. Pat. Nos. 6,140,087; 6,136,594; 6,133,028; 6,120,764. See, e.g., Okada (1996) Gene Ther. 3:957-964; Muzyczka (1994) J. Clin. Invst. 94:1351; U.S. Pat. Nos. 6,156,303; 6,143,548 5,952,221, describing AAV vectors. See also U.S. Pat. Nos. 6,004,799; 5,833,993.

Expression vectors capable of expressing proteins in plants are well known in the art, and can include, e.g., vectors from Agrobacterium spp., potato virus X (see, e.g., Angell (1997) EMBO J. 16:3675-3684), tobacco mosaic virus (see, e.g., Casper (1996) Gene 173:69-73), tomato bushy stunt virus (see, e.g., Hillman (1989) Virology 169:42-50), tobacco etch virus (see, e.g., Dolja (1997) Virology 234:243-252), bean golden mosaic virus (see, e.g., Morinaga (1993) Microbiol Immunol. 37:471-476), cauliflower mosaic virus (see, e.g., Cecchini (1997) Mol. Plant Microbe Interact. 10:1094-1101), maize Ac/Ds transposable element (see, e.g., Rubin (1997) Mol. Cell. Biol. 17:6294-6302; Kunze (1996) Curr. Top. Microbiol. Immunol. 204:161-194), and the maize suppressor-mutator (Spm) transposable element (see, e.g., Schlappi (1996) Plant Mol. Biol. 32:717-725); and derivatives thereof.

The invention provides a transformed cell comprising a nucleic acid of the invention. The cells can be mammalian (such as mouse or human), insect (such as Spodoptera frugiperda, Spodoptera exigua, Spodoptera littoralis, Spodoptera litura, Pseudaletia separata, Trichoplusia ni, Plutella xylostella, Bombyx mori, Lymantria dispar, Heliothis virescens, Autographica californica and other insect, particularly lepidopteran and coleopteran, cell lines), plant, bacterial, yeast, and the like. Techniques for transforming and culturing cells are well described in the scientific and patent literature; see, e.g., Weiss (1995) Methods Mol. Biol. 39:79-95, describing insect cell culture in serum-free media; Tom (1995) Methods Mol. Biol. 39:203-224; Kulakosky (1998) Glycobiology 8:741-745; Altmann (1999) Glycoconj. J. 16:109-123; Yanase (1998) ActaVirol. 42:293-298; U.S. Pat. Nos. 6,153,409; 6,143,565; 6,103,526.

Alignment Analysis of Sequences

The nucleic acid sequences of the invention include genes and gene products identified and characterized by analysis using the exemplary nucleic acid and protein sequences of the invention, including SEQ ID NO:1 and SEQ ID NO:2. For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters are used unless alternative parameters are designated herein. The sequence comparison algorithm then calculates the percent sequence identity for the test sequence(s) relative to the reference sequence, based on the designated or default program parameters. A “comparison window”, as used herein, includes reference to a segment of any one of the number of contiguous positions selected from the group consisting of from 25 to 600, usually about 50 to about 200, more usually about 100 to about 150 in which a sequence may be compared to a reference sequence of the same number of contiguous positions after the two sequences are optimally aligned.

Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Natl. Acad. Sci. USA 85:2444 (1988), by computerized implementations of these algorithms (CLUSTAL, GAP, BESTFIT, FASTA, and TFASTA in the Wisconsin Genetics Software Package, Genetics Computer Group, 575 Science Dr., Madison, Wis.), or by manual alignment and visual inspection.

In one aspect, a CLUSTAL algorithm, such as the CLUSTAL W program, is used to determine if a nucleic acid or polypeptide sequence is within the scope of the invention; see, e.g., Thompson (1994) Nuc. Acids Res. 22:4673-4680; Higgins (1996) Methods Enzymol 266:383-402. Variations can also be used, such as CLUSTAL X, see Jeanmougin (1998) Trends Biochem Sci 23:403-405; Thompson (1997) Nucleic Acids Res 25:4876-4882. CLUSTAL W program, described by Thompson (1994) supra, in the methods of the invention used with the following parameters: K tuple (word) size: 1, window size: 5, scoring method: percentage, number of top diagonals: 5, gap penalty: 3.

Another algorithm is PILEUP, which can be used to determine whether a polypeptide or nucleic acid has sufficient sequence identity to SEQ ID NO:1 or SEQ ID NO:2 to be with the scope of the invention. This program creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive alignment method of Feng & Doolittle, J. Mol. Evol. 35:351-360 (1987). The method used is similar to the method described by Higgins & Sharp, CABIOS 5:151-153 (1989). The following parameters are used with PILEUP in the methods of the invention: default gap weight (3.00), default gap length weight (0.10), and weighted end gaps.

Another example of an algorithm that is suitable for determining percent sequence identity (i.e., substantial similarity or identity) in this invention is the BLAST algorithm, which is described in Altschul (1990) J. Mol. Biol. 215:403-410. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul (1990) supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues, always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. In one embodiment, to determine if a nucleic acid sequence is within the scope of the invention, the BLASTN program (for nucleotide sequences) is used incorporating as defaults a wordlength (W) of 11, an expectation (E) of 10, M=5, N=4, and a comparison of both strands. For amino acid sequences, the BLASTP program uses as default parameters a wordlength (W) of 3, an expectation (E) of 10, and the BLOSUM62 scoring matrix (see, e.g., Henikoff (1989) Proc. Natl. Acad. Sci. USA 89:10915).

Antibodies

The invention provides antibodies that specifically bind to the polypeptides of the invention, e.g., the exemplary SEQ ID NO:1. These antibodies can be used, e.g., to isolate the polypeptides of the invention, to identify the presence of aminotransferases, and the like. To generate antibodies, polypeptides or peptides (antigenic fragments of SEQ ID NO:1) can be conjugated to another molecule or can be administered with an adjuvant. The coding sequence can be part of an expression cassette or vector capable of expressing the immunogen in vivo (see, e.g., Katsumi (1994) Hum. Gene Ther. 5:1335-9). Methods of producing polyclonal and monoclonal antibodies are known to those of skill in the art and described in the scientific and patent literature, see, e.g., Coligan, CURRENT PROTOCOLS IN IMMUNOLOGY, Wiley/Greene, NY (1991); Stites (eds.) BASIC AND CLINICAL IMMUNOLOGY (7th ed.) Lange Medical Publications, Los Altos, Calif.; Goding, MONOCLONAL ANTIBODIES: PRINCIPLES AND PRACTICE (2d ed.) Academic Press, New York, N.Y. (1986); Harlow (1988) ANTIBODIES, A LABORATORY MANUAL, Cold Spring Harbor Publications, New York.

Antibodies also can be generated in vitro, e.g., using recombinant antibody binding site expressing phage display libraries, in addition to the traditional in vivo methods using animals. See, e.g., Huse (1989) Science 246:1275; Ward (1989) Nature 341:544; Hoogenboom (1997) Trends Biotechnol. 15:62-70; Katz (1997) Annu. Rev. Biophys. Biomol. Struct. 26:27-45. Human antibodies can be generated in mice engineered to produce only human antibodies, as described by, e.g., U.S. Pat. No. 5,877,397; 5,874,299; 5,789,650; and 5,939,598. B-cells from these mice can be immortalized using standard techniques (e.g., by fusing with an immortalizing cell line such as a myeloma or by manipulating such B-cells by other techniques to perpetuate a cell line) to produce a monoclonal human antibody-producing cell. See, e.g., U.S. Pat. Nos. 5,916,771; 5,985,615.

It will be readily apparent to one skilled in the art that various substitutions and modifications may be made to the invention disclosed herein without departing from the scope and spirit of the invention. It is understood that the examples and aspects described herein are for illustrative purposes only and that various modifications or changes in light thereof will be suggested to persons skilled in the art and are to be included within the spirit and purview of this application and scope of the appended claims.

### EXAMPLES

The following examples are offered to illustrate, but not to limit the claimed invention.

### Example 1

### Culturing of Bacteria

A sulfur-metabolizing thermophilic archaebacterium, Pyrococcus horikoshi (deposited at JAPAN Collection of Microorganism, RIKEN, Accession No: JCM9974) was cultured as described below.

13.5 g of salt, 4 g of Na₂SO4, 0.7 g of KCl, 0.2 g of NaHCO₃, 0.1 g of KBr, 30 mg of H₃BO₃, 10 g of MgCl₂-6H₂O, 1.5 g of CaCl₂, 25 mg of SrCl₂, 1.0 ml of resazurin solution (0.2 g/l), 1.0 g of yeast extract and 5 g of bactopeptone were dissolved in 11 of water. Then the solution was adjusted to be pH 6.8 and then sterilized under pressure.

Next, dry and heat -sterilized elemental sulfur was added to the solution up to 0.2%. This medium was made anaerobic by saturating with argon, and then JCM9974 was inoculated to the medium. To confirm that the medium became anaerobic, Na₂S solution was added to the medium to see that no pink coloring of resazurin solution resulted from Na₂S in the liquid medium. Then, JCM9974 was cultured in the above liquid medium at 95° C. for 2 to 4 days.

### Example 2

### Preparation of Chromosomal DNA

The chromosomal DNA of JCM9974 was prepared by the following methods.

After culturing of JCM9974, cells were collected by centrifugation at 5,000 rpm for 10 min. The cells were washed twice with 10 mM Tris (pH 7.5)-1 mM EDTA solution, and then sealed into InCert Agarose (FMC) block. The block was treated in a solution containing 1% N-lauroyl sarcosine and 1 mg/ml protease K so that chromosomal D nucleic acid was separated and prepared in the agarose block.

### Example 3

### Construction of Library Clone Containing Chromosomal DNA

The chromosomal DNA obtained in Example 2 was partially digested with restriction enzyme HindIII, and fragments with a length of approximately 40 kb were prepared by means of agarose gel electrophoresis.

Using T4 ligase, the DNA fragments were ligated with Bac vector pBAC108L (Stratagene) and pFOS1 (Stratagene) both of which had been completely digested with restriction enzymes HindIII.

When the former vector pBAC108L was used, the ligated DNA was immediately introduced into E. coli by electroporation.

When the latter vector pFOS1 was used, the ligated DNA was packaged by GIGA Pack Gold (Stratagene) into 1 phage particles in a test tube. Then, E. coli was infected with the particles, thereby introducing DNA into E. coli.

Antibiotic, chloramphenicol-resistant E. coli populations obtained by these methods were designated as BAC and Fosmid library, respectively. Clones appropriate for covering chromosomal DNA of JCM9974 were selected from these libraries and clone alignment was performed.

### Example 4

### Sequencing of BAC or Fosmid Clone

DNA was recovered from each of the aligned BAC and Fosmid clones. The recovered DNA was fragmented by ultrasonication. The fragmented DNA was subjected to agarose gel electrophoresis, and 1 kb and 2 kb-long DNA fragments were recovered. These DNA fragments were inserted into HincII restriction enzyme sites of pUC118 plasmid vectors so that 500 shotgun clones were produced per BAC or Fosmid clone.

Nucleotide sequences of each shot gun clone were determined using Perkin Elmer 373 or 377 (manufactured by ABI, automatic device for reading nucleotide sequences). The nucleotide sequences obtained from each shot gun clone were combined and edited using SEQUENCHER™ (software for automatically combining nucleotide sequences). Therefore, the whole nucleotide sequences of each BAC or Fosmid clone were determined.

### Example 5

### Identification of Aromatic Amino Acid Aminotransferase Gene

The nucleotide sequences of each BAC or Fosmid clone determined in Example 4 were analyzed by a large-scale computer. Thus a gene (SEQ ID NO: 2) encoding aromatic amino acid, aminotransferase was identified.

### Example 6

### Construction of Expression Plasmid

To construct restriction enzyme sites (Ndel and BamHI) before and after a structural gene region, 2 types of DNA primers as shown below were synthesized. PCR was performed using these primers to introduce restriction enzyme sites before and after the structural gene.

| | |
|---|---|
| Upper primer | |
| 5′-TTTTGTCGACTTACATATGGCGCTAAGTGACAGA-3 ′ | SEQ ID NO:3 |
| Lower primer | |
| 5′-TTTTGGTACCTTTGGATCCTTAACCAAGGATTTAAACTAG-3 ′ | SEQ ID NO:3 |

The fragments amplified by PCR were completely digested with restriction enzymes (Ndel and BamHI) at 37° C. for 2 hours, thereby isolating structural genes and the genes were purified.

pET11a (Novagen) was cleaved with restriction enzymes Ndel and BamHI and then purified. Then, the products were allowed to react in the presence of the above structural gene and T4 ligase at 16° C. for 2 hours to be ligated. Next, part of the ligated DNA was introduced into competent cells of E.coli XL 1-BlueMRF′, thereby obtaining colonies of transformants. Expression plasmids were isolated from the obtained colonies, and then purified by the alkaline method.

### Example 7

### Expression of Recombinant Genes

The competent cells of E. coli (E. coli BL21 (DE3), Novagen) were thawed and 0.1 ml of the thawed cells was transferred into a Falcon tube. 0.005 ml of an expression plasmid solution was added to the cells. The mixture was allowed to stand on ice for 30 min, and then subjected to heat shock at 42 for 30 sec. 0.9 ml of SOC medium was added to the mixture, followed by shaking culture at 37° C. for 1 hour. An appropriate quantity of the culture product was inoculated over a 2YT agar plate containing ampicillin and cultured overnight at 37° C., thereby obtaining transformants.

The transformants were cultured in a 2YT medium (21) containing ampicillin until absorption at 600 nm reached 1. Then, IPTG (Isopropyl-b-D-thiogalactopyranoside) was added to the medium followed by culturing for another 6 hours. After culturing, the cells were collected by centrifugation at 6,000 rpm for 20 min.

### Example 8

### Purification of Thermostable Enzymes

The collected cells were frozen and thawed at −20° C. Next, alumina in a volume twice as that of the cells and 1 mg of DNase were added to the cells, disrupting the cells. 5 volumes of 10 mM Tris-hydrochloric acid buffer (pH 8.0) was added to the disrupted cells, thereby obtaining a suspension. The thus obtained suspension was heated at 85 μl for 30 min, followed by centrifugation at 11,000 rpm for 20 min, allowing the supernatant to adsorb to HiTrapQ column (Pharmacia). Then, elution was performed with an NaCl concentration gradient, so that active fractions were obtained. Further, the obtained active fraction solution was applied to a HiLoad 26/60 SUPERDEX200™ pg gel filtration column (Pharmacia), thereby obtaining purified enzymes.

### Example 9

### Measurement of Physical and Chemical Properties of Enzyme

(1) Chemical properties of Enzymes

Determination of protein-coding regions based on nucleotide sequence analysis and N-terminal amino acid sequence analysis revealed that this enzyme comprises 388 residues. Further, the result of SDS polyacrylamide electrophoresis conducted on this enzyme showed that the molecular weight of this enzyme is 44,000 Da. Furthermore, gel filtration analysis using a G2000SWXL™ (Toso) column found that this enzyme has a homodimeric subunit structure. Moreover, the result of isoelectric focusing of this enzyme revealed that the isoelectric point of this enzyme is 5.2.

(2) Amino Acid Group Transfer Reaction

Enzyme reaction was conducted under conditions of pH 8.0 and 25□ using 2-ketoglutaric acid as an amino group receptor and using two types of substrate as amino group donors. Then, kinetic parameters of each substrate were compared. When an acidic substrate (aspartic acid) was used as an amino group donor, malate dehydrogenase was coupled to the reaction. Next, a change in the amount of NADH was traced with a change in absorbance at 340 nm, and then kinetic parameters, Kcat and Km values were measured.

When hydrophobic substrate (phenylalanine) was used as an amino group donor, the amount of reaction product (phenylpyruvic acid) was traced with a change in absorbance at 280 nm, and then Kcat and Km values were measured.

| Substrate | Kcat/s⁻¹ | Km/M | Kcat/Km/s−1M⁻¹ |
|---|---|---|---|
| aspartic acid 2-ketoglutaric | 0.18 | 105 < 0.001 | 1.7 |
| acid | | | |
| phenylalanine 2-ketoglutaric | 12 | 1.2 < 0.001 | 1.0 × 104 |
| acid | | | |

As shown in Table 1, Kcat of this enzyme for phenylalanine and aspartic acid was 12 and 0.18 sec⁻¹(25° C., pH 8.0), respectively; the Kcat/Km value for the same was 1.0×10⁴ and 1.7 sec-1 M⁻¹, respectively. Therefore, it was shown that this enzyme is an aminotransferase having higher catalytic activity for an aromatic amino acid, e.g. phenylalanine than that for a non-aromatic amino acid, e.g. aspartic acid.

(3) Optimum Temperature and Optimum pH

Optimum temperature and optimum pH were measured as described below using L-cysteic acid and 2-ketoglutaric acid as substrates. Optimum temperature was determined according to the temperature dependence of the Kapp value which was determined by varying the reaction temperature from 30° C. to 98° C. in 50 mM phosphate buffer (pH 6.5), tracing an increase in absorbance at 412 nm resulting from reduction of 5,5′-Dithiobis (2-nitrobenzoic acid) (DTNB), finding Kapp value from the initial velocity.

Optimum pH was determined according to pH dependence of the Kapp value which was determined under the measurement conditions as described above by maintaining the reaction temperature at 90° C., and varying the pH of an enzyme reaction solution from 3.4 to 7.5, finding Kapp.

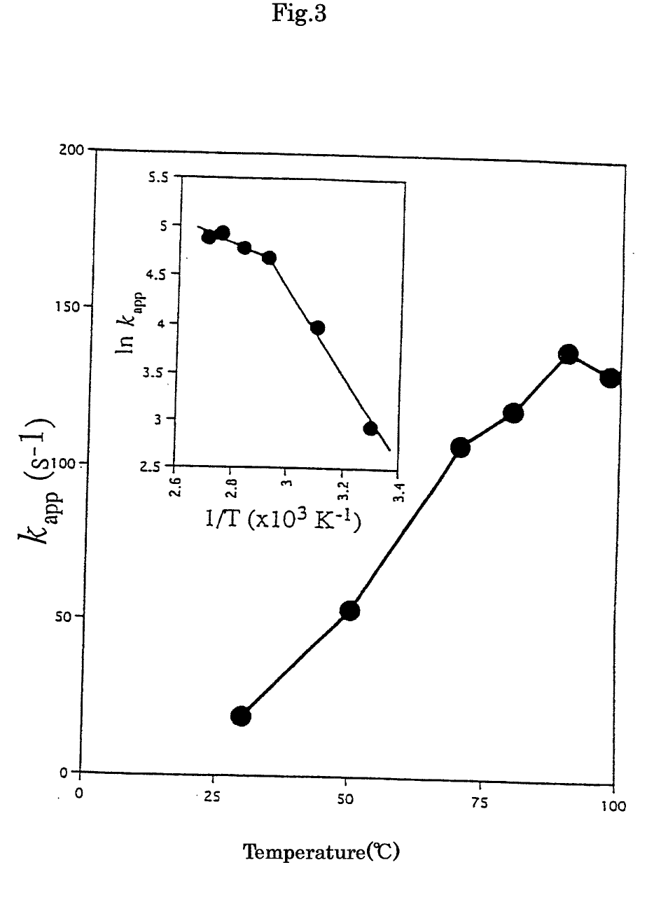

FIG. 3 shows the results. As shown in FIG. 3, when L-cysteic acid and 2-ketoglutaric acid were used as substrates, Kapp value increased as the temperature rose, and peaked at 90° C. At this time, Kapp value was 1.39×10² sec⁻¹ (pH 6.5, 90° C.). Thus, the optimum temperature and the optimum pH of this enzyme was found to be 90° C. and 6.0, respectively.

(4) Thermal Stability

Thermal stability was analyzed by measurement of residual activity after heating and with a differential scanning calorimeter (DSC).

To measure residual activity after heating, this enzyme (0.1 mg/ml) was heated for a certain period of time at 95° C. and at 110° C. in 20 mM phosphate buffer pH 6.5) and quenched. Then, residual activity was measured.

In measurement with DSC, a DSC (type CSC5100™, Calolimetry Science) was used. Cell temperature was increased from 0 to 125° C. (1 K/min), and then a change in thermal capacity of the enzyme protein in 20 mM phosphate buffer (pH 6.5) was measured. Enzyme concentration employed was 1 mg/ml.

As a result of measurement of residual activity after heating, the enzyme following treatment at pH 6.5 and 95° C. for 6 hours remained stable, or was not deactivated. Further, it was found that the enzyme has a half-life at 110° C. of 30 min.

The results of DSC measurement revealed that the melting temperature (Tm value) was 120.1° C. (at pH 6.5) at which the enthalpy change is 2.4×10³ KJ/mole. Moreover, its denaturation was irreversible.

(5) pH Stability

pH stability was analyzed using a circular dichrograph (CD, type J-720W, JASCO Corporation). The pH of an enzyme solution (0.1 mg/ml) was varied from 1.0 to 13.0, a change in intensity of negative ellipicity[q] at 25° C. was measured, so that pH stability was found.

The results revealed that a-helix content of this enzyme at 25° C. and pH 6.5 is 40%, and the enzyme remains stable over a wide pH range from 4 to 11 for 24 hours or more. The results from (4) and (5) suggest that this enzyme shows extremely high thermal stability and pH stability.

Industrial Applicability

The present invention provides aminotransferases which remain stable at high temperature and over a wide pH range. The aminotransferases of this invention are useful as a catalyst for aminotransferase reaction under severe conditions. Particularly, the aminotransferase of this invention is useful as a catalyst of aminotransferase reaction using aromatic amino acid as a substrate, since the aminotransferase has very high aminotransferase activity for aromatic amino acid. Aminotransferase reaction using the aminotransferase of this invention can yield amino acid derivatives with high optical purity.

Furthermore, the present invention provides a gene and nucleic acids for encoding the aminotransferases of this invention. The nucleic acids of this invention are useful in production of the aminotransferase of this invention. That is, the protein of this invention can be produced in large quantity by integrating the nucleic acids of this invention into an expression vector, and introducing the vector into a host cell for expression.

All the documents cited in this specification are incorporated into the specification as references in their entirety.

What is claimed is:

1. An isolated enzyme comprising an aminotransferase activity comprising the following properties:
(a) the enzyme has molecular weight of between about 43,000 Da and about 45,000 Da, or, has an isoelectric point of between about 5.0 and 5.4; and,
(b) the enzyme comprises an aminotransferase activity and exhibits higher aminotransferase activity when an aromatic amino acid is used as an amino group donor rather than when a non-aromatic amino acid is used as an amino group donor.

2. The isolated enzyme of claim 1, wherein the enzyme retains its aminotransferase activity at temperatures over about 90° C.

3. The isolated enzyme of claim 1, wherein the optimum aminotransferase activity is at a temperature of about 90° C.

4. The isolated enzyme of claim 1, wherein the enzyme has aminotransferase activity in conditions comprising a pH of between about pH 4 to about pH 11.

5. The isolated enzyme of claim 1, wherein the optimum aminotransferase activity is at a pH of about pH 6.

6. The isolated enzyme of claim 1, wherein the enzyme maintains its activity after exposure to treatment at about pH 6.5 and 95° C. for about 6 hours.

7. The isolated enzyme of claim 1, wherein the enzyme remains stable at about pH 4 to about pH 11 and about 25° C. for 24 hours or more.

8. The isolated enzyme of claim 1, wherein the enzyme has a melting temperature at about pH 6.5 at about 120.1° C. where molar enthalpy change is about 2.4×103 KJ/mole.

9. The isolated enzyme of claim 1, wherein the enzyme has an a-helix content of about 40% at about pH 6.5 and about 25° C.

10. The isolated enzyme of claim 1, wherein the enzyme has a molecular weight of about 44,000 Da.

11. The isolated enzyme of claim 1, wherein the enzyme has a homodimeric subunit structure.

12. The isolated enzyme of claim 1, wherein the enzyme has an isoelectric point of 5.2.

13. The isolated enzyme of claim 1, wherein denaturation of the enzyme is an irreversible process.

14. 12. The isolated enzyme of claim 1 comprising a sequence as set forth in SEQ ID NO:1.

15. An isolated enzyme comprising aminotransferase activity comprising the following properties:
(a) the enzyme has molecular weight of about 44,000 Da and an isoelectric point of 5.2;
(b) the enzyme exhibits higher aminotransferase activity when an aromatic amino acid is used as an amino group donor rather than when a non-aromatic amino acid is used as an amino group donor, and,
(c) the enzyme has an aminotransferase activity and retains its aminotransferase activity at temperatures over about 90° C.

16. An isolated polypeptide comprising an amino acid sequence as set forth in SEQ ID NO: 1.

17. An isolated polypeptide comprising an amino acid sequence derived from the amino acid sequence of SEQ ID NO: 1 further comprising a deletion, a substitution or an addition of one or more amino acid residues of SEQ ID NO: 1 and having an aminotransferase activity.

18. The isolated polypeptide of claim 16, wherein the substitution is a conservative substitution.

19. An isolated polypeptide comprising an amino acid sequence having at least 85% sequence identity to SEQ ID NO:1, and, the polypeptide has an aminotransferase activity.

20. The isolated polypeptide of claim 19, wherein the sequence identity to SEQ ID NO:1 is at least 90%.

21. The isolated polypeptide of claim 19, wherein the sequence identity to SEQ ID NO:1 is at-least 95%.

22. The isolated polypeptide of claim 19, wherein the sequence identity to SEQ ID NO:1 is at least 98%.

23. The isolated polypeptide of claim 19, wherein the polypeptide has a sequence as set forth in SEQ ID NO:1.

24. An isolated nucleic acid, wherein the nucleic acid encodes a polypeptide as set forth in claim 19.

25. An isolated nucleic acid, wherein the nucleic acid encodes a polypeptide as set forth in SEQ ID NO:1.

26. An expression cassette comprising a nucleic acid comprising a sequence as set forth in claim 25.

27. A transformed cell comprising a heterologous nucleic acid, wherein the nucleic acid comprises a sequence as set forth in claim 24 or claim 25.

28. An array comprising oligonucleotide probes immobilized on a solid support comprising a nucleic acid as set forth in claim 24 or claim 25.

29. An array comprising polypeptides immobilized on a solid support comprising a polypeptide as set forth in claim 1 or claim 19.

30. An isolated antibody that selectively binds to a polypeptide as set forth in claim 1 or claim 19, or a polypeptide encoded by a nucleic acid as set forth in claim 24 or claim 25.

31. The antibody of claim 30, wherein the antibody of a monoclonal antibody.

32. A hybridoma cell line comprising an antibody as set forth in claim 31.

33. A method of making a transformed cell comprising a heterologous aminotransferase nucleic acid or polypeptide comprising introducing a nucleic acid as set forth in claim 24 or claim 25 into a cell, thereby producing a transformed cell.

34. A method of expressing a heterologous nucleic acid sequence in a cell comprising:
(a) transforming the cell with a heterologous nucleic acid sequence comprising a nucleic acid as set forth in claim 24 or claim 25, wherein heterologous nucleic acid sequence comprises a promoter operably linked to the nucleic acid sequence; and
(b) growing the cell under conditions where the heterologous nucleic acid sequence is expressed in the cell.

35. A method of determining whether a test compound specifically binds to an aminotransferase enzyme comprising:
(a) expressing a nucleic acid as set forth in claim 24 or claim 25 under conditions permissive for translation of the nucleic acid to a polypeptide, or, providing a polypeptide as set forth in claim 1 or claim 19;
(ii) contacting the polypeptide with the test compound; and
(iii) determining whether the test compound specifically binds to the polypeptide.


SEQUENCE LISTING (2 sequences)

SEQ ID NO: 1 - protein, 389 aa, Pyrococcus horikoshi
MALSDRLELVSASEIRKLFDIAAGMKDVISLGIGEPDFDTPQHIKEYAKEALDKGLTHYGPNIGLLELREAIAEKLKKQNGIEADPKTEIMVLLGANQAFLMGLSAFLKDGEEVLIPTPAFVSYAPAVILAGGKPVEVPTYEEDEFRLNVDELKKYVTDKTRALIINSPCNPTGAVLTKKDLEEIADFVVEHDLIVISDEVYEHFIYDDARHYSIASLDGMFERTITVNGFSKTFAMTGWRLGFVAAPSWIIERMVKFQMYNATCPVTFIQYAAAKALKDERSWKAVEEMRKEYDRRRKLVWKRLNEMGLPTVKPKGAFYIFPRIRDTGLTSKKFSELMLKEARVAVVPGSAFGKAGEGYVRISYATAYEKLEEAMDRMERVLKERKLV

SEQ ID NO: 2 - DNA, 1170 bp, Pyrococcus horikoshi
atggcgctaagtgacagattagaattagttagtgcttctgaaattagaaagctctttgatattgctgcaggaatgaaggatgttatctccctgggaataggggaacctgattttgatacgcctcaacatattaaggagtatgccaaggaagccctggataagggattgactcattatggtccaaatatagggcttttagagcttagggaagccatagctgaaaagttaaagaagcagaatggcatagaggctgatccaaagacagagataatggtcttattaggtgcgaaccaagctttcttaatgggcctctcggcttttcttaaggatggtgaagaggtattaataccaactccagcctttgtcagctacgcacctgccgttatattggctggaggaaagcccgttgaagtcccaacgtacgaagaggatgaattcaggctaaacgttgatgagcttaaaaagtatgttaccgacaagactagagctttaataataaactcaccgtgtaatccaacgggagcagtgttaactaagaaagatctagaagagatagcggattttgtcgttgaacatgatctaattgtaataagcgatgaagtttatgagcacttcatttacgatgatgctaggcactacagtatagcctccctggatggaatgtttgaaaggacaataaccgttaacggattctcaaagacgtttgcaatgacgggctggaggttgggatttgttgcagcgccttcttggataatagagaggatggtgaagtttcagatgtataacgctacttgtccagtgactttcatacaatacgctgctgctaaagcgttaaaggatgagagaagctggaaagctgttgaagagatgagaaaggagtacgacagaagaagaaagctcgtgtggaagaggcttaacgagatgggactcccaacggtaaagccgaagggtgcattttacatattcccgaggataagggatactgggctaacgagcaagaaattcagcgagctcatgcttaaagaagctagggttgcagtagttccaggtagtgcctttggaaaagccggtgagggatacgtaaggatcagctatgcaacagcttatgagaagcttgaagaggccatggatagaatggaaagggtgttaaaggagaggaagctagtttaa